(19) United States
(12) Patent Application Publication
(10) Pub. No.: US 2026/0092296 A1
(43) Pub. Date: Apr. 2, 2026

(54) Method and/or apparatus for producing a cell inoculum for use in a biomanufacturing or cell culture process

(71) Applicant: St Andrews Pharmaceutical Technology Limited, Henley-On-Thames (GB)

(72) Inventor: William J Henry, St. Andrews (GB)

(21) Appl. No.: 19/096,659

(22) Filed: Mar. 31, 2025

(30) Foreign Application Priority Data
Sep. 27, 2024 (GB) 2414239.0

(51) Int. Cl. C12P 21/00; C12N 5/071; C12N 13/00

(52) U.S. Cl. CPC C12P 21/00; C12N 5/0682; C12N 5/0686; C12N 13/00

(57) ABSTRACT

A method is provided for producing a cell inoculum suitable for use in inoculating a bioreactor in a biomanufacturing or cell culture process. The method includes the steps of taking a cell line sample and undertaking at least one cell passage with said cell line sample to increase the number of cells to form at least a first set of passaged cells. Subsequently said first set and/or one or more subsequent sets of passaged cells is used for optionally inoculating a bioreactor in use. The method further includes the step of applying electrical or electromagnetic stimulus, wave and/or one or more signals to any or any combination of: the cell line sample, the at least first set of passaged cells and/or one or more subsequent sets of passaged cells to create a pre-conditioned cell inoculum. Optionally the pre-conditioned cell inoculum is then used to directly or indirectly inoculate the bioreactor.

A cell inoculum composition suitable for use in a biomanufacturing or cell culture process and comprising a population of epigenetically modified cells is also provided. The cells may, for example, be HEK cells or CHO cells.

This invention relates to a method and/or apparatus for producing a cell inoculum for use in a biomanufacturing or cell culture process.

Although the following description refers almost exclusively to a method and/or apparatus for producing a cell inoculum for inoculating a bioreactor or final stage bioreactor for use in a biomanufacturing or cell culture process, the method can be used to provide pre-conditioning or optimization of cells in a seed train, cell expansion process, biomanufacturing process, cell culture process and/or the like.

Seed train processes, cell expansion processes or processes for providing cell inoculums are often used for the generation of adequate numbers of cells of a particular cell line for the inoculation of a final production bioreactor as part of a biomanufacturing process, or for growing cells as part of a cell culture process. These processes have conventionally been time intensive and cost intensive, involving many cultivation steps taking place for fixed time periods where the cell numbers become larger or are “scaled up” with each cell passage, often in combination with an increase in container size for each cell passage (i.e. a small cell vial, to a flask, to a larger flask, to a small scale bioreactor and then subsequently larger final stage bioreactors). The final production bioreactor for the cell culture is typically inoculated using the largest seed train/cell expansion/cell inoculum passage undertaken in the processes. Alternatively, some of the cell passages may take place in the final production bioreactor using a series of dilution steps.

Conventionally, in order to optimize the abovementioned processes, parameters such as cell growth, uptake of substrates and production of metabolites all have to be calculated and optimized for each cell passage stage. However, even with the current optimization techniques, the process of cell expansion remains an expensive and time-consuming part of any biomanufacturing or cell culture process. Furthermore, as the cell expansion processes becomes scaled up, it becomes more difficult and expensive to optimize the same.

EP3802778 discloses the ability to increase productivity of cell cultures during the expression phase in a final stage, large vessel bioreactor process by applying pulsed electromagnetic signals to the same. As this process has typically been applied to the final large scale vessels having bioreactor capacities of approximately 2000-20,000 Litres, there can be known difficulties associated with ensuring that the pulsed electromagnetic signals are applied consistently and uniformly throughout such a large volume of bioreactor fluid; metallic bioreactors cannot be used due to interference issues with the pulsed electromagnetic signals and therefore plastic large bioreactors have to be sourced. This can result in the seed train process not being as optimal as required and is still time consuming and expensive; and there are problems of isolating a single large bioreactor in a large bioreactor plant from other bioreactors when applying the pulsed electromagnetic signals.

It is therefore an aim of the present invention to provide a method and/or apparatus for producing a cell inoculum for use in inoculating a bioreactor in a biomanufacturing process or cell culture process that overcomes the abovementioned problems.

It is an aim of the present invention to provide a method and/or apparatus for optimizing a biomanufacturing process and/or for optimizing a seed train or cell expansion process for use in a biomanufacturing process.

It is a further aim of the present invention to provide a method and/or apparatus for the optimization of a cell culture process and/or for optimizing a seed train or cell expansion process for use in a cell culture process.

According to a first aspect of the present invention there is provided a method for producing a cell inoculum suitable for use in inoculating a bioreactor in a biomanufacturing or cell culture process, said method including the steps of taking a cell line sample and undertaking at least one cell passage with said cell line sample to increase the number of cells to form at least a first set of passaged cells; and subsequently using said first set and/or one or more subsequent sets of passaged cells for optionally inoculating a bioreactor in use; and wherein said method further includes the step of applying an electrical or electromagnetic stimulus, such as an applied electromagnetic field or a transmitted electromagnetic wave and/or one or more stimulus signals to any or any combination of: the cell line sample, the at least first set of passaged cells and/or one or more subsequent sets of passaged cells to create a pre-conditioned cell inoculum, and optionally then using the pre-conditioned cell inoculum to directly or indirectly inoculate the bioreactor. In a preferred aspect, the stimulus applied is in the form of a pulsed electromagnetic wave (PEMW) signal.

In one aspect the pre-conditioned cell inoculum is the final product and therefore the method is directed to a method for producing a cell inoculum, which can optionally be used for one or more other biomanufacturing or cell culture processes.

In one aspect the method includes the step of inoculating the bioreactor with the pre-conditioned cell inoculum in a biomanufacturing or cell culture process.

Preferably the bioreactor is a final stage bioreactor (such as for example a final production bioreactor in a biomanufacturing process), a production vessel and/or the like.

Preferably the step of applying an electrical or electromagnetic stimulus, field or wave and/or one or more signals is sufficient so as to impart a metabolic change to the cells in the pre-conditioned cell inoculum, such that the metabolic change in the cells in the pre-conditioned cell inoculum is hereditary and is retained or permanently retained in the cells inoculated into the bioreactor or used in a subsequent biomanufacturing or cell culture process without having to apply or reapply the electrical or electromagnetic stimulus signal, field or wave to the bioreactor or to the subsequent process. In an example, we have found that in particular when the stimulus is applied in the form of a pulsed electromagnetic wave (PEMW), such metabolic changes occur and are hereditary and are retained, including being permanently retained, in the cell inoculum. A population of the cells in the cell inoculum become epigenetically modified cells, having altered genetic expression, and having epigenetic markers which distinguish them from untreated cells. The Applicants have surprisingly found that metabolic changes to the cells in the pre-conditioned inoculum, such as for example changes or enhancements to the chromosomal proteins, histone acetylation, ribosomal activity and/or gene expression, as a result of the application of the electrical or electromagnetic stimulus signal, field or wave, appear to be permanent and hereditary and result in these changes being passed on to later cells within whatever process the pre-conditioned cell inoculum is subsequently used for. This means that there is no need to apply or re-apply the electrical or electromagnetic stimulus signal, field or wave to optimize growth or product output at a later stage in the biomanufacturing or cell culture process, such as to the final stage bioreactor. This has the advantage that it is easier to undertake and control the electrical or electromagnetic treatment in relation to a relatively small volume of the cells (passaged cells) and it is not necessary to repeat the application of the electrical or electromagnetic treatment to the same seed train or cell expansion process once a set of cells has been treated. Thus, the pre-conditioning of cells in a biomanufacturing and/or cell culture process using the present invention is advantageous and unexpected and provides an increased product output from the process. Thus the method as herein described is a less time consuming and more cost effective method of optimizing a seed train or cell expansion process, and therefore optimizing a biomanufacturing process or cell culture process in which the passaged/expanded cells are used, by performing the present invention on a relatively small scale compared to trying to optimize the process when the process has been scaled up. In addition, the Applicant has found that cell proliferation, gene expression, ribosomal activity, metabolic activity and final product output is greater and cell death is reduced in the pre-conditioned cell inoculum compared to when the cells are not pre-conditioned.

Since the application of the electrical or electromagnetic stimulus signal, field or wave is undertaken on the cell line and/or at an earlier stage of the seed train/cell expansion process compared to conventional processes, the signals can be applied through a container which is more commonly formed of a material that the electrical or electromagnetic stimulus signal, field or wave can more easily pass through, such as for example, glass or plastic. This overcomes problems associated with applying electrical or electromagnetic stimulus signals, fields or waves through larger scale containers which are more typically formed of metal, such as for example large metal or stainless steel bioreactors.

Furthermore, when a bioreactor is inoculated with cells, it is often in a cell production plant with other bioreactors present. It is often difficult to isolate the electrical or electromagnetic stimulus signal, field or wave to just one bioreactor. However, this problem of isolation of the bioreactor is overcome using the method of the present invention.

Thus, the present invention produces a pre-conditioned cell inoculum that can optimize a seed train process, a cell expansion process, a biomanufacturing process and/or a cell culture process by causing cell changes or pre-conditioning of the cells much earlier in the process than with the prior art.

Accordingly, in a further aspect, the present invention also provides a cell inoculum composition suitable for use in a biomanufacturing or cell culture process comprising a population of epigenetically modified cells, wherein the cell inoculum is obtainable by a process comprising the step of applying an electrical or electromagnetic stimulus, wave and/or one or more signals to any or any combination of: a cell line sample, a first set of passaged cells derived from the cell line sample by a cell passage step to increase the number of cells, and/or one or more subsequent sets of passaged cells derived from the first set of passaged cells by one or more cell passage step(s) to increase the number of cells; wherein the epigenetically modified cells exhibit an increase in protein productivity in comparison to untreated cells, an increase in ribosomal activity in comparison to untreated cells, or both. Suitably, the epigenetically modified cells exhibit an increase in protein productivity of at least 30% or more in comparison to untreated cells, an increase in ribosomal activity of at least 50% or more in comparison to untreated cells, or both.

In a further aspect, the present invention also provides a cell inoculum composition, suitable for use in a biomanufacturing or cell culture process, which composition comprises a population of epigenetically modified cells, wherein the cell inoculum is obtainable by, or obtained by, a process comprising the step of applying an electrical or electromagnetic stimulus, field or wave and/or one or more signals to any or any combination of: a cell line sample, a first set of passaged cells derived from the cell line sample by a cell passage step to increase the number of cells, and/or one or more subsequent sets of passaged cells derived from the first set of passaged cells by one or more cell passage step(s) to increase the number of cells; wherein the epigenetically modified cells have one or more of the following characteristics compared to untreated cells: increased cell proliferation, increased chromosomal protein expression, increased ribosomal biogenesis or ribosomal activity, increased histone acetylation or histone methylation, increased metabolic activity, or reduced cell death.

The cell inoculum composition thus comprises a population of conditioned or altered cells, which cells have significantly different epigenetic properties or markers, compared to the untreated or naturally occurring cells. The terms conditioned or pre-conditioned cell inoculum may be useful interchangeable to refer to an inoculum composition comprising such altered cells.

Suitably, at least 50% or more of the cells in the cell inoculum composition are epigenetically modified cells. Additionally, or alternatively, in some aspects at least 60% or more of the cells in the cell inoculum composition are epigenetically modified cells, preferably at least 70% or more, preferably at least 80% or more, more preferably at least 90% or more, most preferably 95% or more of the cells in the cell inoculum composition are epigenetically modified cells. In one aspect, substantially all, or about 100%, of the cells in the cell inoculum composition are epigenetically modified cells. It has been found that this is possible by applying the stimulus, as described, to a small volume of cells, for example to a cell line sample, following resuscitation of the cells from a cell bank. For example, in one aspect. the stimulus may be applied whilst the cell culture is in a healthy, exponential growth state, but prior to any, or any significant, normal cell expansion process or phase. Typically, such a sample may comprise a volume of 1 litre or less. In one aspect, application of the stimulus is preferably applied to a cell line sample before passaging the cell culture as part of a cell expansion process, although the stimulus may for example be applied after one of more passages whilst the volume of the cell culture remains small, for example suitably less than 2 litres, or less than 5 litres, or less than 10 litres.

In a preferred aspect, the stimulus applied is in the form of a pulsed electromagnetic wave (PEMW) signal. Thus, in a preferred aspect, the invention provides a cell inoculum composition, suitable for use in a biomanufacturing or cell culture process, which composition comprises a population of epigenetically modified cells, wherein the cell inoculum is obtainable by, or obtained by, a process comprising the step of applying an electromagnetic stimulus in the form of a pulsed electromagnetic field or wave to any or any combination of: a cell line sample, a first set of passaged cells derived from the cell line sample by a cell passage step to increase the number of cells, and/or one or more subsequent sets of passaged cells derived from the first set of passaged cells by one or more cell passage step(s) to increase the number of cells; wherein the epigenetically modified cells exhibit an increase in protein productivity in comparison to untreated cells, an increase in ribosomal activity in comparison to untreated cells, or both. Suitably, the epigenetically modified cells exhibit an increase in protein productivity of at least 30% or more in comparison to untreated cells, an increase in ribosomal activity of at least 50% or more in comparison to untreated cells, or both.

In one aspect, the epigenetically modified cells may have one or more of the following characteristics compared to untreated cells: increased cell proliferation, increased chromosomal protein expression, increased ribosomal biogenesis or ribosomal activity, increased histone acetylation or histone methylation, increased metabolic activity, or reduced cell death. For example, in some aspects the epigenetically modified cells may be characterized as exhibiting an increase in cell proliferation of at least 30%, compared to untreated cells. Additionally or alternatively, in some aspects the epigenetically modified cells may be characterized as exhibiting an increase in chromosomal protein expression of at least 40% compared to untreated cells. Additionally or alternatively, in some aspects the epigenetically modified cells may be characterized as exhibiting an increase in ribosomal biogenesis or ribosomal activity of at least 20% compared to untreated cells. Additionally or alternatively, in some aspects the epigenetically modified cells may be characterized as exhibiting an increase in metabolic activity of at least 40% compared to untreated cells. Additionally or alternatively, in some aspects the epigenetically modified cells may be characterized as exhibiting a reduction in cell death of at least 25% compared to untreated cells.

Preferably the cell line, the one or more cell passages and/or the bioreactor can include suspended cells (i.e. cells suspending in a liquid or cell media liquid) and/or adherent cells (i.e. cells grown on a solid substrate or solid cell media).

In one aspect the cell line sample, first set of passaged cells and/or one or more subsequent sets of passaged cells includes any or any combination of cells suspended in a fluid; adherent cells; or a plurality of cells of the cell line and a liquid, cell media, substrate and/or carrier agent.

In one aspect the point in the passages, seed train or cell expansion process at which the electrical or electromagnetic stimulus, field or wave and/or signals are applied at least partly depends on the volume or amount of the liquid, cell media, carrier agent and/or substrate including the cells (i.e. the sample or cell passage set) which is being exposed to the electrical or electromagnetic stimulus, field or wave and/or signals. For example, if there is less than 1 L volume of liquid including the cells, the electrical or electromagnetic stimulus, field or wave and/or signal treatment may be provided on the third set of passaged cells, or earlier, for example on the second or first set of passaged cells, or before this on the cell line sample itself. Suitably the largest volume of liquid including the cells the electrical or electromagnetic stimulus, field or wave and/or signal treatment may be provided on, in one example, is less than or equal to 200 L or less than or equal to 100 L or less than or equal to 50 L volume of liquid including the cells. In one aspect, the volume is preferably smaller than this. For example, pulsed electromagnetic field (PEMW) signals may be applied to the cell inoculum, for example to a container or vessel comprising the cell inoculum, wherein the cell inoculum has a volume in a single vessel of 10 litres or less; or a volume in a single vessel of 2 litres or less, or a volume in a single vessel of 1 litre or less.

In one aspect the point in the seed train/cell passages/cell expansion at which the electrical or electromagnetic stimulus, field or wave and/or signals are applied to the cell line and/or set(s) of passaged cell is any or any combination of: before the first cell passage; during the first cell passage; after the first cell passage; at a last shake flask step in the method/seed train/cell expansion; at the last cell passage of the method/seed train/cell expansion; at the last cell passage undertaken within an incubator or heat step used in the method/seed train/cell expansion; at or after the third or fourth cell passage of the method/seed train/cell expansion; at or after the third or fourth cell passage undertaken within an incubator used in the method/seed train/cell expansion and/or the like.

In one example the largest volume of liquid or carrier agent in which the cell line or plurality of cells is provided to form the pre-conditioned cell inoculum is less than or equal to 200 L, although in one preferred aspect smaller volumes are employed as described above. However, other aspects can be provided with large volumes of liquid or carrier agent.

In one aspect, one or more cell passages take place in an incubator and the electrical or electromagnetic stimulus, field or wave and/or signals are applied before, during and/or after an incubator cell passage step.

In one aspect the apparatus that applies the electrical or electromagnetic stimulus, field or wave and/or signals process is provided in, is associated with and/or is integral with the incubator.

Preferably 1-4 cells passages take place in the incubator as part of the method/seed train/cell expansion process.

Preferably the incubator warms cells to approximately 37 degrees C. in one example.

In one aspect the one or more cell passages take place at room temperature (i.e. 20-22° C.) and/or atmospheric pressure.

Preferably reference to cell passages, cell expansion process or seed train process herein are terms that can be used interchangeably and generally refer to a process whereby the number of cells is expanded via at least one, and preferably a plurality, of cell passages.

In one aspect the electrical or electromagnetic stimulus, field or wave and/or signals, preferably a pulsed electromagnetic wave (PEMW) signal, is applied to the cell line between about 0-12 or about 0-24 hours before the at least one or first cell passage, and preferably between about 0-48 hours, about 0-72 hours or about 0-96 hours, about 24 hrs, about 48 hrs, about 72 hrs or about 96 hrs before the at least one or first cell passage. It will be appreciated that at least some stimulus will be applied i.e. the duration cannot be zero. In one example, a suitable application is for around 2 days (about 48 hours).

In one aspect, a cell inoculum composition comprising a population of epigenetically modified cells is produced by applying pulsed electromagnetic wave (PEMW) signals to a cell line sample following resuscitation of the cells from a cell bank. This may, for example, be prior to a normal cell expansion passaging process. It is a feature of the present invention that pulsed electromagnetic wave (PEMW) signals may be applied at an early stage in the process, and that the resulting cell inoculum composition comprising a population of epigenetically modified cells has cellular characteristics, or epigenetic markers, that are both permanent and heritable.

It is surprising and unexpected that the use of electrical or electromagnetic stimulus, field or wave and/or signals on the cells according to the present invention causes significant and substantial metabolic changes, such as for example to the cell's gene expression, metabolic activity and/or ribosomal activity and that these changes are permanent and persist in subsequent cell passages and/or throughout the remainder of the seed train, cell expansion process, biomanufacturing process and/or cell culture process.

In one aspect the electrical or electromagnetic stimulus, field or wave and/or signals are applied to the cells for about 12 hours, about 24 hours, about 48 hours, about 72 hours, about 96 hours; or between about 0-12 hours, about 0-24 hours, about 0-48 hours, about 0-72 hours or about 0-96 hours. It will be appreciated that at least some stimulus will be applied i.e. the duration cannot be zero. In one aspect the electrical or electromagnetic stimulus, field or wave and/or signals could be applied to the cells for up to 10 days if required.

In some aspects, the changes in the cell's gene expression as a result of the pre-conditioning of the present invention favours protein productivity and/or cell viability. For example, the Applicants have found that the pre-conditioning as disclosed herein yields a yield increase in protein productivity in the pre-conditioned cell inoculum of at least 15%, at least 20%, at least 21%, at least 22%, at least 23%, at least 24%, at least 25%, at least 26%, at least 27%, at least 28%, at least 29%, or about 30% in comparison to untreated cells, as determined by human IgG enzyme-linked immunosorbent assay (ELISA) as a result of use of the disclosed method and/or apparatus. A human IgG enzyme-linked immunosorbent assay (ELISA) is used to specifically detect and quantify human IgG in cell culture supernatants. The assay employs anti-human whole IgG, raised in goat, for capture, and an anti-human Fc antibody conjugated to horseradish peroxidase (HRP), also raised in goat, for detection. Upon addition of the substrate o-phenylenediamine dihydrochloride (OPD), HRP catalyzes a colorimetric reaction that produces a signal directly proportional to the concentration of IgG in the sample. The IgG titer is determined by measuring absorbance at 492 nm, and the concentration is derived from a standard reference curve with known IgG concentrations. Additionally or alternatively, the preconditioning as disclosed herein yields an increase in ribosomal activity of cells in the pre-conditioned cell inoculum of at least at least 25%, at least 30%, at least 35%, at least 40%, at least 41%, at least 42%, at least 43%, at least 44%, at least 45%, at least 46%, at least 47%, at least 48%, at least 49%, or about 50% in comparison to untreated cells, as determined by human IgG enzyme-linked immunosorbent assay (ELISA) (as above) as a result of use of the disclosed method and/or apparatus. A cell inoculum composition comprising a population of epigenetically modified cells wherein the cells comprise at least a 30% yield increase or more in protein productivity and/or at least a 50% increase or more in ribosomal activity, compared to untreated cells, is therefore an aspect of the present invention.

In one aspect the cell line and/or the at least first set of passaged cells only need to be treated once using the electrical or electromagnetic stimulus, field or wave and/or signal treatment. This was found to be sufficient to bring about an improvement and/or optimization of the process. Thus, in an example, treatment once only by application of a pulsed electromagnetic wave (PEMW) signal to a cell inoculum composition is a preferred feature of the invention. It was also found to be sufficient to provide a hereditary change in the cells undergoing the electrical or electromagnetic stimulus, field or wave and/or signal treatment.

In one aspect there is no treatment required for the final stage bioreactor cells or subsequent biomanufacturing or cell culture processes using electrical or electromagnetic stimulus, field or wave and/or signals. However, it will be appreciated that electrical or electromagnetic stimulus, field or wave and/or signals could also be applied to the final stage bioreactor cells and/or subsequent processes if required.

In one aspect the electrical or electromagnetic stimulus, field or wave and/or signal treatment is applied at two or more different times and/or stages prior to inoculation of the bioreactor or final stage bioreactor and/or use in a subsequent process.

In one aspect an increase in glucose, nutrients, liquid, cell media, cell media substrate, support substrate, and/or growth media is provided in the seed train, cell expansion process, biomanufacturing process and/or cell culture process to support the increased rate of cell proliferation caused by the exposure of the cells to the electrical or electromagnetic stimulus, field or wave and/or signal treatment. Thus, in one example, cellular growth is optimized during the cell passages to take into account the increase in cell growth. However, it is to be noted that an increase in cell growth, protein production, metabolic changes and/or the like is achieved even without media/reagent optimization.

In one aspect the glucose, nutrients, liquid, cell media, cell media substrate, support substrate and/or growth media is supplemented as recommended by any or any combination of the manufacturers; cell line providers; automatically via a computer system, in-line monitoring device and/or one or more sensors; and/or the like.

Preferably an amount of glucose and/or nutrients in the carrier agent, liquid, cell media, growth media and/or culture media used in the method of the present invention is optimised in order to optimize cell viability, protein productivity and/or recombinant product; and/or is maintained at or above 4 g/Litre.

Preferably the glucose within the liquid, cell media, cell media substrate, support substrate and/or growth media is adjusted so as to be kept at a level equal to or above 4 g/Litre. In one example, this is deemed sufficient to maintain cell viability, protein productivity and/or recombinant product during the process.

In one aspect the method is optimised to allow for optimised cell viability, optimised recombinant product and/or optimised protein production at each stage or cell passage of the method.

In one example, growth reagents used in the liquid, cell media, growth media, cell media substrate and/or support substrate may include any or any combination of glucose, amino acids, vitamins, protein, insulin, phosphates and/or the like.

In one example, the method for producing the cell inoculum, the seed train or the cell expansion process consists of 2-3 cell passages.

In one example, the method for producing the cell inoculum, the seed train or the cell expansion process consists of up to 12 cell passages.

Preferably the method includes shaking, agitating and/or vibrating the cells at one or more stages in the method using suitable shaking, agitating and/or vibrating means or devices to produce the pre-conditioned cell inoculum.

Preferably the step of applying electrical or electromagnetic stimulus, field or wave and/or signal treatment includes applying any or any combination of pulsed electromagnetic wave (PEMW) signals, PULZAR™ (St Andrews Pharmaceuticals Technology Ltd, UK), one or more signals or waves in the electromagnetic spectrum, Radio Frequency (RF) signals or waves, microwaves, using one or more coils, magnets, Alternating Current (AC) and/or the like.

The term “pulsed electromagnetic wave (PEMW) signals” used herein define a sequence or pattern of signals or waves in the electromagnetic spectrum range that change in amplitude from a base line to a higher or lower value, followed by a return to the base line or a return substantially to the base line. Further preferably the change in signal amplitude is rapid and transient and occurs in a repeating sequence. In one example, the base line represents an absence of electromagnetic signals or waves being emitted from an electromagnetic signal source or transmission means. Preferably the base line is considered to be a rest or relaxation period for the cells and/or pulsed electromagnetic signals or waves.

Preferably the PEMW signals are pulsed or intermittent radio wave or RF signals.

Preferably the method takes place in-vitro.

Preferably the cell line used in the method or in the cell inoculum composition is a mammalian cell line, plant cell line, insect cell line, fungi cell line, bacterial cell line and/or the like. Examples of possible cell lines used could include: 3T3 cells (a mouse fibroblast cell line derived form a spontaneous mutation in cultured mouse embryo tissue; fibroblast cell line; cancer cell line; A549 cells (derived from a cancer patient lung tumor); HeLa cells (human cell line isolated from cervical patient Henrietta Lacks); HEK 293 cells (derived from human fetal cells); Huh7 cells (hepatocyte derived carcinoma cell line); Jurkat cells (a human T lymphocyte cell line isolated from a case of leukemia); OK cells (derived from female North American opossum kidney cells); Ptk2 cells (derived from male long-nosed potoroo epithelial kidney cells); Vero cells (a monkey kidney cell line that arose by spontaneous immortalisation); yeast (Saccharomyces cerevisiae); fungal cells, including Pichia cells, CHO cells (Chinese Hamster Ovary cells); PER C6 cells (derived from human embryonic retinal cells transformed with the Adenovirus Type 5 (Ad5) E1A and E1B genes); Primary T cells and/or the like.

In one aspect, the population of epigenetically modified cells in the cell inoculum comprise HEK cells, HEK 293 cells, or CHO cells. The epigenetic modification in such epigenetically modified cells compared to untreated or unconditioned cells comprises one or more of: increased ribosomal biogenesis, increased cytoplasmic ATP production, increased histone acetylation, or increased histone methylation.

In one aspect, the cell inoculum composition may comprise a population of epigenetically modified HEK cells, wherein the epigenetically modified HEK cells exhibit an increase in protein productivity in comparison to untreated cells, an increase in ribosomal activity in comparison to untreated cells, or both. Suitably, the epigenetically modified cells exhibit an increase in protein productivity of at least 30% or more in comparison to untreated cells, an increase in ribosomal activity of at least 50% or more in comparison to untreated cells, or both.

In a further aspect, the cell inoculum composition may comprise a population of epigenetically modified HEK cells, wherein the epigenetically modified HEK cells show increased ribosomal biogenesis, increased cytoplasmic ATP production, increased histone methylation or histone acetylation, or decreased mitochondrial activity compared to untreated HEK cells.

In one preferred aspect, the HEK cells are HEK-293 cells.

In one aspect, the cell inoculum composition may comprise a population of epigenetically modified CHO cells, wherein the epigenetically modified CHO cells exhibit an increase in protein productivity in comparison to untreated cells, an increase in ribosomal activity in comparison to untreated cells, or both. Suitably, the epigenetically modified cells exhibit an increase in protein productivity of at least 30% or more in comparison to untreated cells, an increase in ribosomal activity of at least 50% or more in comparison to untreated cells, or both.

In another aspect, the cell inoculum composition may comprise a population of epigenetically modified CHO cells, wherein the epigenetically modified CHO cells show increased ribosomal biogenesis, increased cytoplasmic ATP production or increased histone methylation or histone acetylation, or increased mitochondrial activity compared to untreated CHO cells.

Preferably the step of applying electrical or electromagnetic stimulus, field or wave and/or signals at the cells takes place at room temperature (such as for example 20-22° C.) or takes place in an incubator that can be set at temperatures above room temperature (such as for example at 37° C.).

In one aspect the step of applying electrical or electromagnetic stimulus, field or wave and/or signals takes place for a pre-determined time period. In one example, the time for which the cells receive the applying electrical or electromagnetic stimulus, field or wave and/or signals is approximately 48 hours. However, it will be appreciated that longer or shorter time periods could be used if required.

In one aspect the pre-determined time period for which the cells receive the electrical or electromagnetic stimulus, field or wave and/or signals is approximately at or between approximately 1-4 hours, and further preferably approximately 3-4 hours. However, it will be appreciated that longer or shorter time periods could be used if required. For example, in one aspect the pre-determined time period can be up to 12 hours, or up to 24 hours, up to 48 hours, up to 72 hours up to 96 hours or between 5-10 days.

In one aspect the minimum pre-determined time period for which the cells receive the applying electrical or electromagnetic stimulus, field or wave and/or signals is approximately 48 hours.

Preferably the electrical or electromagnetic stimulus, field or wave and/or signals used in the method of the present invention are generated by one or more electronic devices.

Preferably the one or more electronic devices include transmission means or devices for generating and/or transmitting the electrical or electromagnetic stimulus, field or wave and/or signals therefrom in use.

Preferably the one or more electronic devices include an antenna to help transmit or broadcast the electrical or electromagnetic stimulus, field or wave and/or signals therefrom in use.

For example, the one or more electronic devices includes a transmitter and antenna to emit and radiate the electromagnetic stimulus, field or wave and/or signals in use.

Preferably the one or more electronic devices are powered using an alternating current (AC) power supply.

Preferably the transmission means or devices includes one or more electronic transmission chips, the one or more electronic transmission chips arranged to generate, emit and/or transmit one or more pulsed electromagnetic signals in use.

In one aspect reference to the transmission means/device or one or more electronic transmission chips could include one or more transmitters, at least one transmitter and at least one receiver, one or more transceivers, one or more antenna and/or the like. Thus, in one example, the pulsed electromagnetic signals could be transmitted from a central location or a master transmitter and/or antenna and could be received by one or more remote and/or slave receivers and/or transceivers for subsequent re-transmission or emission therefrom.

In one aspect the electronic device has a single transmission means or electronic transmission chip. Such a single transmission means or electronic transmission chip is sufficient to provide a pulsed electromagnetic signal to small cell culture container or vial in one example. In one exemplary aspect, a single transmission means or electronic transmission chip is provided attached or integrated into a flask or small scale bioreactor containing one or more suspended cells. Such a bioreactor operates by stirring the suspension with a stirrer, and as such the cells suspended, typically in media, will pass by the transmission means or electronic transmission chip and thus be exposed to the pulsed electromagnetic signal of the present invention.

In one aspect the electronic device has two or more transmission means or electronic transmission chips. Preferably the two or more transmission means or electronic transmission chips are arranged a pre-determined spaced distance apart from each other in the electronic device.

Preferably the pre-determined spaced distance apart is such so as to provide one or more items or material being pulsed with the electromagnetic pulsed signals sufficient signal strength to achieve a desired effect (i.e. of changing gene expression, of increasing cell viability, of increasing protein production and/or the like) and/or to provide an even or substantially even distribution of electromagnetic radiation/signals in use.

Preferably the electronic device has a plurality of transmission means or electronic transmission chips arranged in a pre-determined pattern and/or array.

Whilst a single transmission means and antenna is sufficient to provide the advantageous properties of the invention, it has been found that having a plurality of transmission means allows the pulsed electromagnetic signal to be delivered across a broader range of surface areas whilst still maintaining a maximal effect. Applicants have found that having a transmission means or electronic transmission chip evenly distributed such that there is at least one chip per 18.5 cm² provides sufficient coverage for the optimal effect.

In some aspects, the apparatus comprises one or more transmission means or electronic transmission chips. In some aspects, the apparatus comprises 2, 3, 4, 5, 6, 7, 8, 9, or 10 or more transmission means or electronic transmission chips.

In some aspects, there is one transmission means or electronic transmission chip per approximately 105 to 115 cm² of a surface of the housing of the apparatus or a surface of an item as defined herein, and preferably approximately 110 cm² of a surface of the housing of the apparatus or a surface of an item as defined herein.

In some aspects, there is one transmission means or electronic transmission chip per approximately 50 to 60 cm² of a surface of the housing of the apparatus or a surface of an item as defined herein, and preferably approximately 55 cm² of a surface of the housing of the apparatus or a surface of an item as defined herein.

In some aspects there is one transmission means or electronic transmission chip per approximately 25 to 30 cm² of a surface of the housing of the apparatus or a surface of an item as defined herein, and preferably approximately 27.5 cm² of a surface of the housing of the apparatus or a surface of an item as defined herein.

In some aspects there is one transmission means or electronic transmission chip per approximately 15 to 20 cm² of a surface of the housing of the apparatus or a surface of an item as defined herein, and preferably approximately 18.5 cm² of a surface of the housing of the apparatus or a surface of an item as defined herein.

In some aspects, there is one transmission means or electronic transmission chip per approximately 10 to 15 cm² of a surface of the housing of the apparatus or a surface of an item as defined herein, and preferably approximately 12.2 cm² of a surface of the housing of the apparatus or a surface of an item as defined herein.

The items as defined herein or in which the at least one cell passage and/or pulsed electromagnetic signal treatment takes place preferably comprise one or more cell culture plates, vials, flasks, shake flasks, wave bags, roller bottles, small scale biorcactors, intermediate bioreactors, large scale bioreactors, wave bag reactor and other vessels known to the skilled person. For example, standard laboratory microplates as defined below, T25, T75, T125, T175, T225, and larger cell culture plates. The one or more transmission means or electronic transmission chips are set a pre-determined space apart according to the surface area of such vessels placed on the device in use, and/or based upon a surface of the housing of the apparatus.

In an exemplary aspect, six transmission means or electronic transmission chips are provided in the apparatus upon which a standard laboratory microplate is positioned. These standard laboratory microplates are provided as 6-well, 12-well, 24-well, 48-well, 96-well, 384-well, and 1536 well plates (and above). These microplates are generally of a standardized size, with dimensions of approximately 128 mm in length by 85 mm in width, thus giving the plate a surface area of approximately 110 cm². Thus, in the exemplary aspect, the 6 transmission means or electronic transmission chips can be evenly spaced to provide an optimal pre-determined space for providing any of these plate types with a pulsed electromagnetic signal according to the present invention. In one example, the electronic device includes six transmission means or electronic transmission chips. Preferably the six transmission means or electronic transmission chips are arranged a pre-determined distance apart from each other such that when a 24 well plate is located in, on or relative to the electronic device in use, each transmission means or chip is able to emit sufficient strength electromagnetic signals and/or is directed to 4 wells of the plate.

Further preferably the transmission means or transmission chip is located adjacent to the 4 wells of the 24 well plate in a central or substantially central position.

In one aspect, where more than one transmission means or electronic transmission chip is required, the spacing of the plurality of transmission means or electronic transmission chips must be optimised. In order to achieve an optimal pre-determined space between each transmission means or electronic transmission chips, the transmission means or electronic transmission chips should be positioned at a distance equal or substantially equal to half the wavelength of the electromagnetic radiation frequency being used. Preferably this distance should be considered to be relevant in any plane of orientation or two or more transmission means or electronic transmission chips being used together as part of the apparatus. For example, if the wavelength is 12.4 cm, the transmission chips should be placed approximately 6.2 cm apart to produce an optimal electromagnetic stimulus, field or wave, wave and/or one or more signals when in use.

In one example, the pre-determined spaced distance=wavelength/2.

In one example, the pre-determined spaced distance in the X-axis and/or Y-axis is half the wavelength between each transmission means or electronic transmission chip in an evenly spaced grid. Such an arrangement minimises the risk of destructive interference.

In one aspect the electronic device includes a housing and the one or more transmission means or transmission chips are located in said housing.

Preferably the housing includes at least one flat or planar surfaces to allow the housing to be located in a stable manner with respect to the one more items receiving the pulsed electromagnetic signals in use. Alternatively, the housing can include one or more curved or non-planar surfaces to allow the housing to be located in a stable manner with respect to one or more items receiving the pulsed electromagnetic signals in use.

In one example, at least one surface of the housing includes one or more recesses for the location of the one or more items receiving the pulsed electromagnetic signals in use.

In one aspect the housing includes a base surface for allowing the housing to be supported directly or indirectly on a surface in use. Further preferably the housing includes an upper surface opposite to the base surface. Preferably the upper surface is the surface on which the one or more items receiving the pulsed electromagnetic signals can be positioned in use.

In one example, the one or more items can be cell culture vials, cell culture plates, flasks and/or the like known to the person skilled in the art in which eukaryotic cells may be cultured.

In one aspect the electronic device and/or housing is attachable to an external surface of a container, vial, flask, reactor vessel, incubator, incubator shelf and/or the like. For example, the electronic device and/or housing can be attachable via one or more attachment means or device including any or any combination of one or more screws, nuts and bolts, magnets, ties, clips, straps, inter-engaging members, adhesive, welding and/or the like. In one example, the electronic device and/or housing is provided internally of the incubator.

In one example, one or more vibration means, stirrers and/or shaking apparatus is required during the seed train process to vibrate or shake the cells in use.

Preferably the upper surface of the housing and/or the distance between the transmission means and the one or more items receiving the pulsed electromagnetic signals when located on, in or relative to the housing or electronic device in use is approximately 25 cm or less, 20 cm or less, 15 cm or less, 10 cm or less or 5 cm or less. Further preferably the distance is approximately 1 cm.

Preferably the pulsed electromagnetic signals are provided in a pre-determined sequence of pulses.

In one aspect the electronic device is arranged to transmit the pulsed electromagnetic signals at a frequency in the range of approximately 2.2-2.6 GHz and, further preferably the pulsed electromagnetic signals are transmitted at a frequency of approximately 2.4 GHz+/−50 MHz or more preferably 2.45 GHz+/−50 MHz.

In one aspect the electronic device is arranged to transmit the pulsed electromagnetic signals at a frequency within the range of the industrial, scientific and medical radio frequency band (ISM band) of 2.4 to 2.4835 GHz, preferably 2.45 GHz+/−50 MHz. In one example, pulsed electromagnetic radio wave signals are preferred.

Preferably the pulsed electromagnetic signals are pulsed at a frequency of approximately 50 Hz or less, further preferably approximately 25 Hz or less, and yet further preferably approximately 15 Hz or less.

Preferably each pulse of the pulsed electromagnetic signals lasts for between approximately 1 ms-20 ms. Further preferably each pulse lasts for approximately 1 ms.

Preferably the time period between pulses (also referred to as the “rest period” or “relaxation period”) is approximately 66 ms or less.

Preferably the duty cycle of the pulsed electromagnetic signals is less than 2%.

In one aspect the transmission power provided by each transmission means or chip in the electronic device is 2 dBm-+4 dBm, approximately 1 mW, approximately 2 mW, approximately 2.5119 mW or approximately 1 mW-2.5119 mW. In one example, the power is 2 mW-20 mW or 3 dBm to 13 dBm.

In one aspect the pre-determined frequency of the pulsed electromagnetic signals is approximately 2.2-2.6 GHz, 2.4 GHz+/−50 MHz or 2.45 GHz+/−50 MHz, the pre-determined pulse rate is approximately 15 Hz or has a duty cycle of less than 2%, and the pre-determined power is +2 dBm-+4 dBm, 1 mW, 2 mW, 2.5119 mW or approximately 1 mW-2.5119 mW, or 2 mW-20 mW.

Preferably the pulsed electromagnetic signals are transmitted using Gaussian Frequency Shift Keying (GFSK) between 0.45 and 0.55.

Preferably the pulsed electromagnetic signals are radio frequency (RF) data signals.

Preferably the pulsed electromagnetic signals is a digital sequence of pulsed electromagnetic signals.

In one aspect the electronic device includes power supply means for supplying electrical power to the device in use. Preferably the power supply means includes a mains electrical power supply, one or more batteries, power cells, one or more rechargeable batteries, electrical generator means and/or the like.

In one aspect the electronic device includes control means for controlling operation of the electronic device and/or transmission means in use.

In one aspect the electronic device includes one or more circuit boards. Preferably the transmission means can be provided on the one or more circuit boards, typically in the form of an integrated circuit, and/or other components, such as for example memory means, are located.

In one aspect the electronic device includes memory means, such as a memory device, data storage device and/or the like.

Preferably the other components of the electronic device includes one or more components required for the selective operation of the apparatus and, when active, the controlled operation of the same to generate the electrical or electromagnetic stimulus, field or wave and/or signals. For example, user selection means can be provided on the device to allow user selection of one or more conditions, operation and/or one or more parameters of the device in use; display means to display one or more settings, options for selection and/or the like.

In one aspect the said further components or power supply means include one or more power cells and the same may all be contained within the housing.

In one aspect the housing of the electronic device is provided in a form which allows the same to be engaged with and/or located with respect to a container in which the material and/or one or more items which is to be exposed to the electrical or electromagnetic stimulus, field or wave and/or signals is located in use.

In one aspect the control means includes an option to allow the user to select any or any combination of the signal frequency, signal strength, signal power, signal pulse rate, time period of signal pulsing, and/or the like of the said pulsed electromagnetic signals. In one aspect the selection of the frequency, strength, power, pulse rate, time period of pulsing, other parameters and/or the like may be made with respect to the particular form of the material and/or one or more items which is to be exposed to the pulsed electromagnetic signals in use, the quantity of said material, the dimensions of the container with respect to which the apparatus is located for use and/or other parameters.

In one aspect the pre-conditioned cell inoculum or at least one set of PEMW treated passaged cells are added to a final bioreactor, or the pre-conditioned cell inoculum or the at least one set of PEMW treated cells undergo one or more passaging steps in a final bioreactor.

According to a further aspect of the present invention there is provided a method for a biomanufacturing process, said method including the steps of: taking a cell line sample and undertaking at least one cell passage with said cell line sample to increase the number of cells to form at least a first set of passaged cells; and subsequently using said first set and/or one or more subsequent sets of passaged cells for inoculating a bioreactor and/or for providing a cell bank, and wherein said method further includes the step of applying an electrical or electromagnetic stimulus, field or wave and/or one or more signals to any or any combination of: the cell line sample, to the at least first set of passaged cells and/or one or more subsequent sets of passaged cells to create a pre-conditioned cell inoculum, subsequently inoculating the bioreactor and/or providing the cell bank with the pre-conditioned cell inoculum. Application of a pulsed electromagnetic wave (PEMW) signal is preferred. A pre-conditioned cell inoculum composition obtained by the above method also forms an aspect of the invention. According to an aspect of the present invention there is also provided a system for producing a cell inoculum suitable for use in inoculating a bioreactor as part of a biomanufacturing or cell culture process, said system including:
container means for the containment of a cell line sample;
passage means to allow at least one cell passage to be undertaken in respect of the cell line sample to increase the number of cells to form at least a first set of passaged cells and subsequently using said first set and/or one or more subsequent sets of passaged cells for optionally inoculating a bioreactor;
means for the application of an electrical or electromagnetic stimulus, field or wave and/or one or more signals to any or any combination of: the cell line sample, the at least first set of passaged cells and/or one or more subsequent sets of passaged cells to create a pre-conditioned cell inoculum, and the system optionally including means for allowing direct or indirect inoculation of a bioreactor using the pre-conditioned cell inoculum. Application of a pulsed electromagnetic wave (PEMW) signal is preferred.

According to a further aspect of the present invention there is provided a system for producing a cell inoculum suitable for use in a biomanufacturing or cell culture process, said system including at least one container for the containment of a cell line and/or at least one set of passaged cells in use; means for emitting an electrical or electromagnetic stimulus, field or wave and/or one or more signals therefrom in use; and wherein said means for emitting an electrical or electromagnetic stimulus, field or wave and/or one or more signals are arranged relative to said at least one container so that the cell line and/or the at least one set of passaged cells can receive the electrical or electromagnetic stimulus, wave and/or one or more signals in use. Application of a pulsed electromagnetic wave (PEMW) signal is preferred.

Preferably the means for emitting the electrical or electromagnetic stimulus, field or wave and/or one or more signals in use include transmission means and an antenna.

Further preferably the transmission means and antenna are arranged to emit PEMW signals in use.

In a further aspect of the invention there is provided apparatus for optimising a change in condition of a product, said apparatus including at least a first container of a size to allow at least a first quantity of a cellular product to be held therein in a fluid or adherent state; and said apparatus includes means to emit an electrical or electromagnetic stimulus, field or wave and/or one or more signals into the said first quantity of the cellular product when in the first container to cause a change in condition of the same; and wherein the means to emit the electrical or electromagnetic stimulus, field or wave and/or one or more signals into the said first quantity of the cellular product is such that the change in condition in the cellular product is inherited and/or maintained during subsequent division and/or replication of the cellular product. Application of a pulsed electromagnetic wave (PEMW) signal is preferred.

According to another aspect of the present invention there is provided a method of applying an electrical or electromagnetic stimulus, field or wave and/or one or more signals to a cell sample including a plurality of cells in such a manner so as to result in a metabolic change, a change in chromosomal markers and/or gene expression in the cells, and wherein the metabolic change, the change in chromosomal markers and/or the change in gene expression persists, is hereditary and/or is permanent in the plurality of cells even after the application of the electrical or electromagnetic stimulus, field or wave and/or one or more signals is stopped. Cells or a cell sample obtained by the above method also forms an aspect of the invention.

Preferably the metabolic change, the change in chromosomal markers and/or gene expression persists for at least a number of cell cycles, cell replication and/or cell divisions of the plurality of cells.

It will be appreciated that the present invention could also have application in any biomanufacturing process, cellular agriculture, as well as biotechnology biomanufacturing or cell culture to name a few non-exhaustive lists of examples.

In a yet further aspect of the invention there is provided a system for causing a change in condition of a bioreaction material, said system including providing a first quantity of the said material and exposing said material to pulsed electromagnetic wave (PEMW) signals generated from one or more modules located in and/or adjacent to said material, and wherein once said change in condition is detected as having occurred, adding to and/or combining said first quantity with a second quantity of the said material to create a combined quantity of said material greater than the first quantity, and enable said change in condition of the material to at least persist in said combined quantity of the said material.

In one aspect further quantities of the said material are added to the previously combined quantity of the material until a required volume of the material is achieved and in which the said change of condition persists.

In one aspect the said addition of the second and subsequent quantities of material occurs in a stepwise manner and/or at predetermined time periods.

Preferably the material is cellular material or cells.

In one aspect the said change in condition of the material spreads throughout the said combined quantity of material without the need for the said combined quantity of material to be exposed to the PEMW signals.

In one aspect the said one or more modules which emit the PEMW signals are located as part of a base onto which a container in which said first quantity of said material is located.

According to one aspect of the present invention there is provided a method for a seed train or cell expansion process for a biomanufacturing or cell culture process, said method including taking a cell line sample and undertaking at least one cell passage with said cell line sample to increase the number of cells to form at least a first set of passaged cells; and subsequently using said first set and/or one or more subsequent sets of passaged cells for inoculating a bioreactor and/or for providing a cell bank in use, and wherein said method further includes the step of applying an electrical or electromagnetic stimulus, field or wave and/or one or more signals to the cell line sample, to the at least first set of passaged cells and/or one or more subsequent sets of passaged cells, prior to optionally inoculating the bioreactor and/or providing the cell bank. Cells, or a cell line sample, or a first set of passaged cells, or one or more subsequent sets of passaged cells, obtained by the above method also forms an aspect of the invention.

### BRIEF DESCRIPTION OF THE DRAWINGS

The invention will now be described with reference to the following drawings; wherein:

FIG. 1a illustrates a conventional seed train process, with the inclusion of the pulsed electromagnetic signal treatment according to the present invention;

FIG. 1b is a schematic of the experimental design for experiment 1;

FIG. 2 is a Volcano Plot of the PEMW samples at 48 hours from experiment 1;

FIG. 3 shows a gene ontology dot plot report for the PEMW samples at 48 hours from experiment 1;

FIG. 4 is a gene heat map of each of the PEMW samples and each of the control samples at 48 hours from experiment 1;

FIG. 5 shows the graphical plot of the average live cells across the three experiments for the control and the PEMW (Pulzar®) results in Experiment 2; and

FIG. 6 is a graphical plot showing the average yield over time for the control and PEMW (Pulzar®) results for Experiment 2.

### DETAILED DESCRIPTION

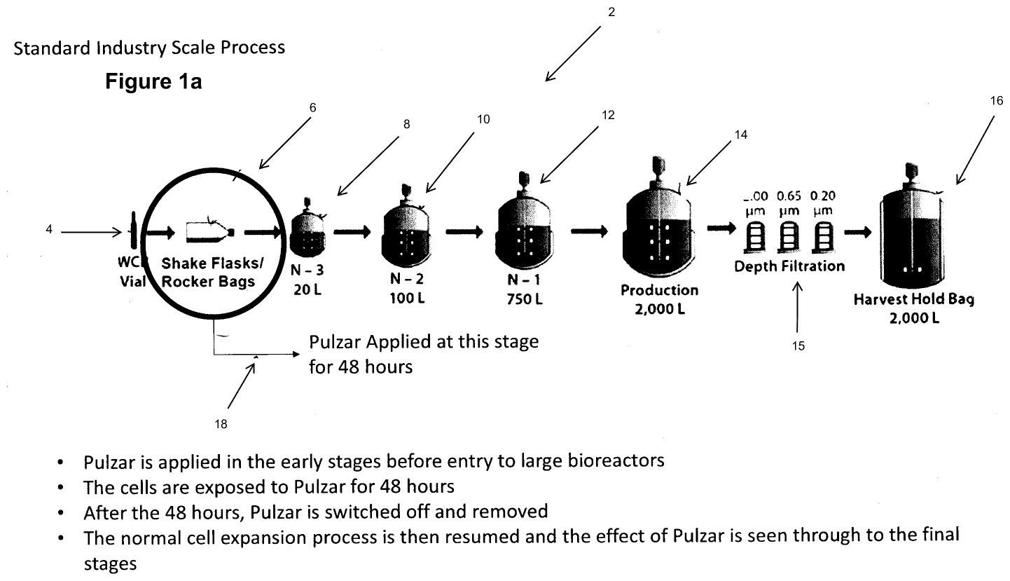

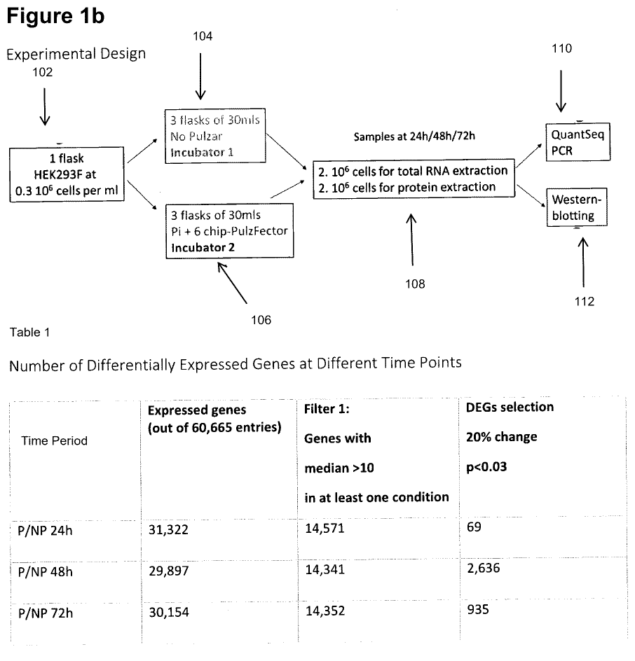

Referring to FIG. 1a, there is illustrated a conventional seed train or cell expansion process 2, wherein a vial 4 containing a cell line for use in a cell culture process is scaled up using the seed train or cell expansion process into increasingly larger sized containers 6, 8, 10, 12, 14 and 16 for each cell passage of the seed train/cell expansion process. For example, the cells from the cell line 4 undergo a first cell passage in a shake flask or a rocker bag 6. The cells then undergo a second cell passage in a 20 L N-3 bioreactor 8, a third cell passage in a 100 L N-2 bioreactor 10, a fourth cell passage in a 750 L N-1 bioreactor 12 and then this cell line is inoculated into a final bioreactor 2000 L production vessel 14 as part of a biomanufacturing process. The cell culture then undergoes depth filtration, as shown by step 15 before being transferred into a 2000 L harvest hold bag 16.

In accordance with the present invention, a pulsed electromagnetic signal treatment using Pulzar® (St. Andrews Pharmaceuticals Ltd, UK) (at a signal frequency of 2.45 GHz and pulsed at a frequency of 15 Hz) is applied to the cells during the first cell passage for 48 hours in the shake flask/rocker bag 6, as shown by arrow 18. The pulsed electromagnetic signal treatment is then removed and the remaining seed train/cell expansion is carried out as per a conventional seed train/cell expansion process. The resulting cells that have been pre-conditioned with the pulsed electromagnetic signal treatment to form a pre-conditioned cell inoculum for subsequent and optional inoculation into a final stage bioreactor.

More particularly, the pulsed electromagnetic signal treatment was applied to a 1 L shake flask for 48 hours. This treatment forms a pre-conditioned inoculum. The cells were seeded at 1×10⁵ cells/ml and were allowed to grow to 1×10⁶ cells/ml, at which point the cell culture was passaged.

The 1 L contents of the shake flask were added to a 20 L vessel containing 9 L of fresh, pre-warmed cell media. The 1:10 dilution with fresh media again gives a seeding density of 1×10⁵ cells/ml. Once the contents of the 10 L vessel reached 1×10⁶ cells/ml (which typically took approximately 48 hours), a second cell passage was undertaken.

The 10 L contents of the previous vessel were then added to a 100 L vessel containing 90 L of fresh, pre-warmed cell media. The cell count is now back to 1×10⁵ cells/ml. The process was repeated until the desired volume of cell culture is achieved.

The Applicants have surprisingly and unexpectedly found that the RNA/DNA changes that occur on exposure of the cells at the first cell passage stage last throughout the seed train/cell expansion process and are present in all the cells in the final stage bioreactor 14 after it has been inoculated with the passaged pre-conditioned cell inoculum. As such, there is no longer a requirement to expose the final stage bioreactor cells to pulsed electromagnetic signals, as was thought necessary in the prior art, in order to result in increased cell protein productivity at this stage of the bioreactor process.

The following experiments described below are used to demonstrate the advantages of the present invention.

### Experiment 1

Experiment 1 was undertaken to demonstrate the difference in gene expression of the RNA of a HEK293F cell line (derived from human foetal cells) following the exposure of the cell line to pulsed electromagnetic (PEMW) signals according to the present invention.

### Methodology of Experiment 1

Referring to FIG. 1b, a flask of cells 102 from the cell line HEK293F at 0.3×10⁶ cells per ml of media were taken and subdivided into three control samples 104 and three PEMW samples 106.

Each control sample comprised three flasks, each with 30 ml of cells and media in the same, as shown by reference numeral 104. The control samples were placed in an incubator 1 at 37 degrees C. No PEMW signals were applied to the control samples. Each PEMW sample comprised three flasks, each with 30 ml of cells and media in the same, as shown by reference numeral 106. The PEMW samples were placed in an incubator 2 and pulsed electromagnetic signals were applied at a frequency of 2.45 GHz and a power of 2.5 mW using a Pulzfector device (St. Andrews Pharmaceuticals Ltd, UK). A Pulzfector device has six electronic chips emitting the pulsed electromagnetic signals.

2×10⁶ cell samples were taken at 24 hrs, 48 hrs and 72 hrs for total RNA extraction and protein extraction to determine the number of differentially expressed genes of the PEMW samples compared to the control samples, as shown by reference numeral 108. A Quantseq PCR method (https://www.lexogen.com/wp-content/uploads/2023/01/015UG009V0271_QuantSeq-3% E2%80%98-mRNA-Seq_2023-01-24.pdf)

was used, as shown by reference numeral 110, to show gene expression changes; and a western blot method (Bourdon et al., Genes Dev. 2005, PMID: 16131611) was used, as shown by reference numeral 112, to separate and identify proteins.

### Results of Experiment 1

The results of Experiment 1 are as follows:

Referring to Table 1, it can be seen that 31,322 differentially expressed genes were found at 24 hrs in the PEMW samples compared to the control samples. Statistical analysis of the differentially expressed genes (DEGs) showed the change in differential expressed genes in the PEMW samples was significant. Table 1 also shows that the number of differential expressed genes did not significantly change in the PEMW samples relative to the control in the 48 hrs and 72 hrs samples compared to the 24 hr samples. This suggests that any gene expression in the cells following PEMW exposure was permanent and not transient. This supports the hypothesis that exposure of a cell line to PEMW in a least one set of passaged cells results in differential gene expression that is maintained to the point of inoculation into the final bioreactor. This removes the problems associated with applying PEMW signals to a large bioreactor.

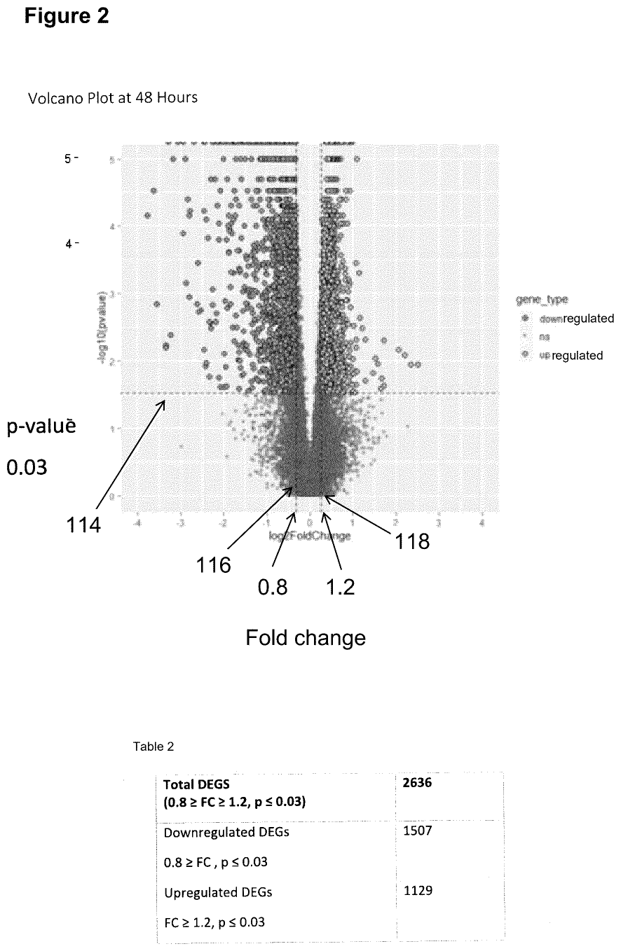

FIG. 2 is a Volcano Plot of the PEMW samples taken at the 48 hour sample time point in experiment 1. A Volcano Plot is a type of scatterplot showing statistical significant (P value on the Y axis) versus magnitude change (fold change on the X axis). It allows quick visual identification of genes with large fold changes that are statistically significant. The dotted horizontal line 114 shows the P value at 0.03. The dotted vertical lines 116, 118 show a 0.8 fold change and 1.2 fold change respectively. The data points on the left show down regulated genes and the data points on the right show up regulated genes. Table 2 shows a summary of the results shown in the Volcano Plot in FIG. 2. It shows that the total number of differentially expressed genes (DEGs) having a fold change greater than or equal to 0.8 and less than or equal to 1.2 that had a statistical probability of greater than or equal to 0.03 was 2636. This comprised 1507 genes that were upregulated and 1129 that were downregulated.

Table 3 shows the changes in mitochondrial proteins in the cell samples exposed to the PEMW signal at 48 hrs. The total number of genes expressed in the QuantSeq dataset that encoded for non-mitochondrial molecules was 28811. Of these, 2380 genes (or 8.26% genes) were regulated/changed following exposure to PEMW signals. The total number of genes expressed in the QuantSeq dataset that encoded for mitochondrial molecules was 1086. Of these, 256 genes (or 23.57%) were regulated/changed following exposure to PEMW signals. This demonstrates that a higher proportion of genes encoding for mitochondrial proteins were changed or controlled following exposure to PEMW signals compared to genes encoding for non-mitochondrial proteins.

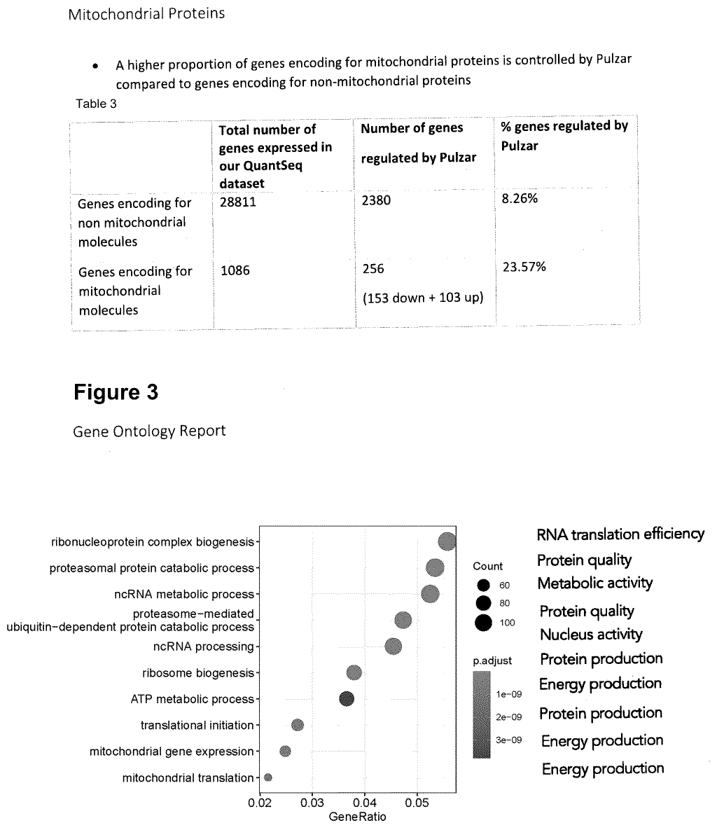

FIG. 3 shows the gene ontology dot plot report for the PEMW samples taken at the 48 hour sample time point in experiment 1. The report shows the function of the genes identified on the left (Y-axis) and the activity in which the genes are involved with on the right. The size of the dots on the plot represent the gene count according to the key, the shading of the dot shows the probability (P-adjust) according to the key and the X axis shows the gene ratio.

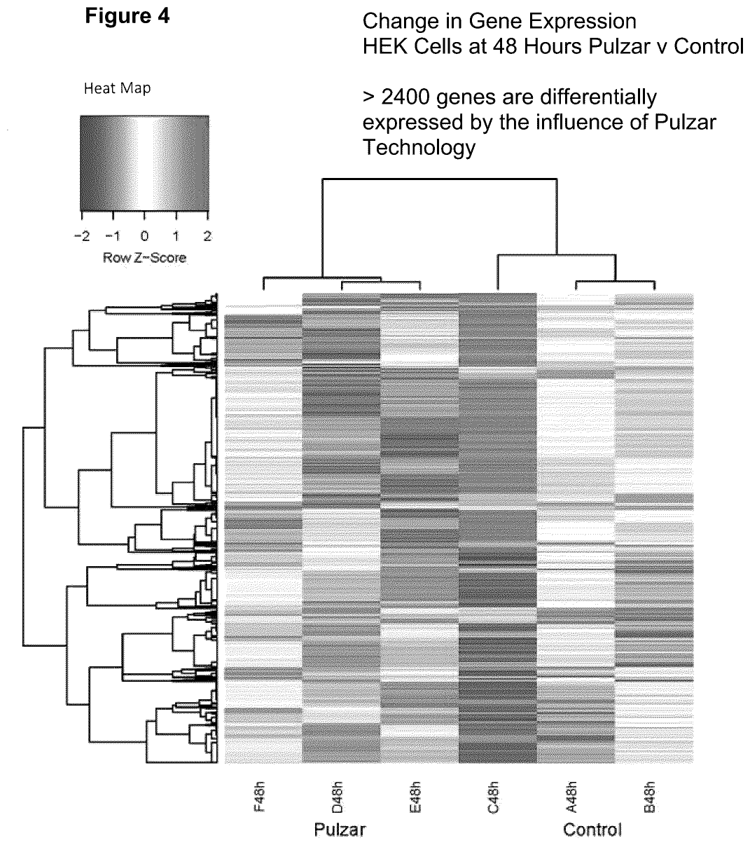

FIG. 4 shows a gene heat map with each of the three PEMW samples and each of the three control samples shown in each column. The colour and intensity of the boxes is used to represent changes (not absolute values) of gene expression. It can be seen that more than 2400 genes of HEK cells are differentially expressed following exposure to PEMW signals compared to the control HEK cell samples.

### Experiment 2

Experiment 2 was undertaken to demonstrate the increase in cell proliferation and the reduction in cell death of recombinant clonal CHO cells exposed to pulsed electromagnetic signals according to the present invention.

### Method

### Establishing and Maintaining the Recombinant Monoclonal CHO Cell Line

Recombinant clonal CHO cells were revived from liquid nitrogen and growth maintained following standard procedures at The Antibody Company, UK (TAC). Briefly, cells were retrieved from storage in liquid nitrogen, thawed and resuspended in 10 mL of TransFX media (Cytiva, UK) supplemented with 4 mM Glutamax™ (Thermo Fisher Scientific, UK) and 0.1% Pluronic F68 (Thermo Fisher Scientific, UK). Cells were centrifuged at 200 g for 5 minutes and the pellet resuspended in 30 mL of TransFX media and transferred to a 125 mL shake flask. The flask was incubated at 37° C., 8.0% CO2, 100 rpm and the cells observed, splitting back into fresh media containing hygromycin (Thermo Fisher Scientific, UK) at 200 μg/mL in shake flasks as required, to maintain their log phase growth. Cells were maintained for 29 days with 8 passages prior to establishing the bioreactors.

### Culture Set Up and Cell Sampling

A small sample of cell suspension was removed from the shake flask and cell density, cell size and viability were measured using the fluorescent AOPI stain with the K2 cellometer.

Based on the results from the K2 count, the appropriate volume of cells was used to seed 35 mL of TFX+200 μg/mL hygromycin to establish a concentration of 1×10⁵ cells/mL. A second count was performed to confirm the correct cell density had been achieved (note: 1×10⁵ cells/mL is outside the accurate range for the K2 cellometer so the values obtained by this second read were for guidance only). At appropriate sampling time points, 1.5 mL sample was taken from each bioreactor, clarified by centrifugation at 1670 g for 5 minutes, and stored at −20° C. for analysis by standard TAC ELISA at a later date. The amount of glucose in the media prior to cell growth was also measured.

The treated shake flask was seated on one Pulzar® device (St. Andrews Pharmaceuticals Ltd, UK) for emitting PEMW signals at frequency 2.45 GHz and power 2.5 mW, with a second Pulzar® device attached to the outside of the shake flask and switched on. The untreated shake flask (Control) was maintained under otherwise identical conditions in a discrete incubator, 3 m distant. Cultures were maintained at 37° C., 8.0% CO2, orbital shakers at 100 rpm for 12 days. The Pulzar® treatment took place for the first two days. After this point, the Pulzar® treatment was switched off and removed.

After treatment each culture was used to set up a production culture as described above. At this point both cultures were relocated to the same incubator to reduce variability in the study.

On days 0, 4, 6, 8 and 10 a 1.5 mL sample was taken from each culture and used to measure cell density, viability, cell size and glucose level of the media. All measurements were performed in duplicate. The remainder of the sample was split into two aliquots and stored at −20° C. for analysis by ELISA at a later date. Cultures were carried out in triplicate.

If glucose levels fell to 22.5 mmol/L or lower, glucose was supplemented to raise levels to approximately 50 mmol/L. Additional glucose was added at day 6 as it had dropped to 8.67 mmol/L in the untreated cultures. This brought the glucose concentration to over 40 mmol/L in both treated and untreated cultures.

The concentration of antibody in each sample taken from each culture run was measured using a standard TAC direct binding ELISA run in triplicate. A standard antibody of known concentration was run as a reference on the ELISA plates.

Samples were diluted 1 in 2 across the ELISA plate, starting at a 1/16 dilution. The reference antibody was diluted 1 in 2 across the ELISA plate from a starting concentration of 1000 ng/ml.

The final data was evaluated further using GraphPad Prism to calculate μg/mL of antibody present, relative to the reference, in the samples during each experiment to determine quantitatively if the Pulzar device was affecting the production of antibody.

More particularly, for the assessment of antibody production by ELISA. The concentration of antibody in each sample taken from both treated and untreated cultures was measured using a standard TAC ELISA and the mean results presented graphically. A standard antibody of known concentration was run as a control on the ELISA plates and to allow calculation of antibody levels. All samples were run in duplicate.

Briefly, the plates were coated with polyclonal anti-human IgG at a concentration of 5 μg/mL (100 μL/well) in bicarbonate coating buffer pH 9.6. The plate was incubated at +4° C. before blocking with 300 μL/well) 2% BSA/PBS for 1 hour at room temperature.

Samples were diluted 1 in 4 across the ELISA plate, starting at a 1/16 dilution. The control antibody was diluted 1 in 4 across the ELISA plate from a starting concentration of 1000 ng/ml. The final volumes were (100 μL/well). The plates were incubated for 1 hour at room temperature. After each plate was washed 4 times in PBS the secondary anti-human Fc specific-HRP conjugate was added at a dilution of 1:600 in 0.1% BSA/PBS (100 μL/well) and the plates were incubated for 1 hour at room temperature in the dark.

After a final wash step as above 100 μL/well of OPD was added. Followed by incubation at room temperature for 20 minutes in the dark, the reaction was stopped using 50 μL/well of sulphuric acid stop solution. The plates were read at 492 nm.

The final data was evaluated further using GraphPad Prism to calculate the concentration of antibody present, relative to a known standard.

### Results of Experiment 2

Table 4 shows the average live cells per ml in the control results from experiment 2.

Table 5 shows the average live cells per ml in the PEMW (Pulzar®) results from experiment 2.

Table 6 shows the average live cells per ml in the control results and the PEMW (Pulzar®) results from experiment 2.

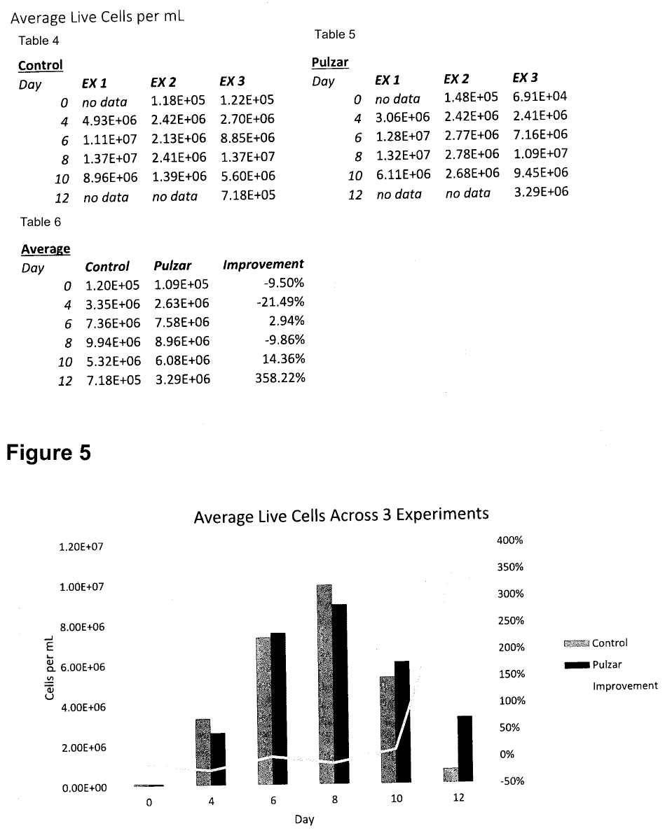

FIG. 5 shows the graphical plot of the average live cells across the three experiments for the control and the PEMW (Pulzar®) results. A significant improvement in the average number of live cells at day 12 is found in the cells exposed to PEMW signals compared to the control cells.

Tables 7-9 show the average cell yields for the control samples and the PEMW (Pulzar® samples) across three separate experiments.

Table 10 shows the average cell yields across an average of all three experiments.

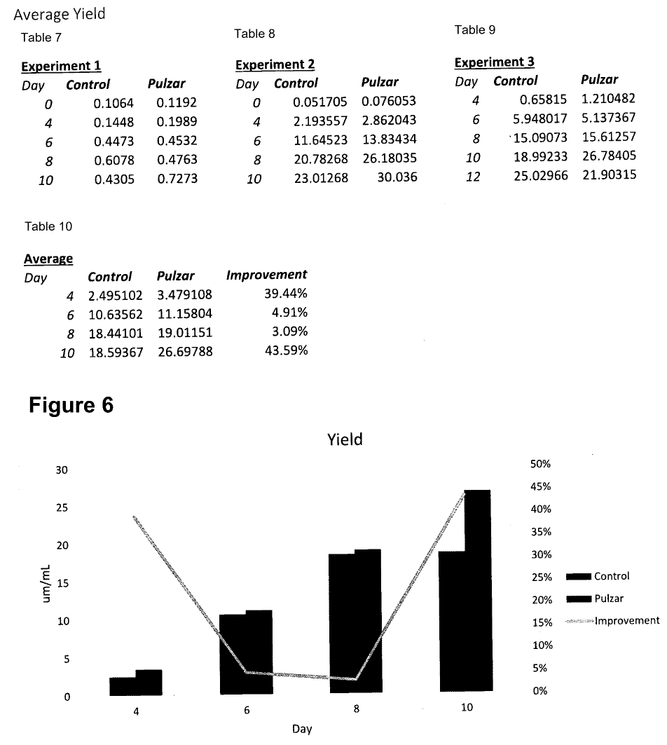

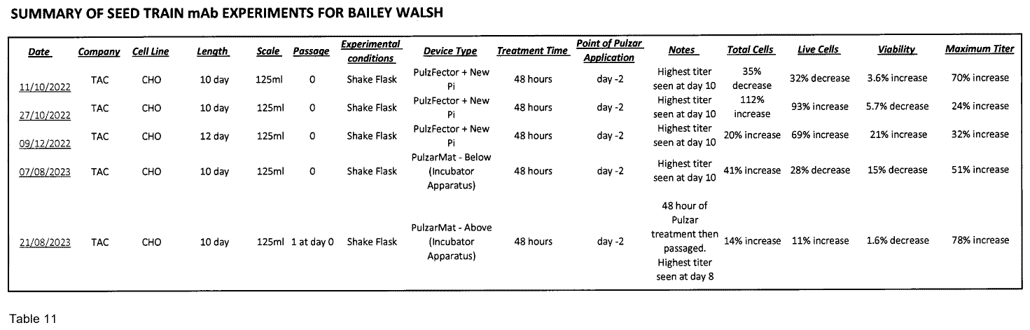

FIG. 6 is a graphical plot showing the average yield over time for the control and PEMW (Pulzar®) results for Experiment 2. A significant improvement in the yield is found at day 10 in the PEMW exposed cells compared to the control cells.

Table 11 shows a summary of the seed train mAb experiments data. This table shows the date at which the experiments took place; the independent company that performed the experiments (The Antibody Company, UK); the cell line used in the experiments; the length of time the experiment took place for; the size of the flasks used in the experiments; the number of passages that took place; the experimental flask used; the type of pulsed electromagnetic signal device used; the length of time the pulsed electromagnetic signals were provided for; the point in the experiment at which the pulsed electromagnetic signals were applied; notes from the experiments; the % total change in the number of cells, the % total change in the number of live cells; the % change in cell viability and the % maximum titer.

Further aspects of the invention may include the following:
Aspect 1: A method for producing a cell inoculum suitable for use in inoculating a bioreactor in a biomanufacturing or cell culture process; said method including the steps of taking a cell line sample and undertaking at least one cell passage with said cell line sample to increase the number of cells to form at least a first set of passaged cells; and subsequently using said first set and/or one or more subsequent sets of passaged cells for optionally inoculating a bioreactor in use; and wherein said method further includes the step of applying an electrical or electromagnetic stimulus, wave and/or one or more signals to any or any combination of: the cell line sample, the at least first set of passaged cells and/or one or more subsequent sets of passaged cells to create a pre-conditioned cell inoculum, and optionally then using the pre-conditioned cell inoculum to directly or indirectly inoculate the bioreactor.
Aspect 2: The method according to Aspect 1, wherein the step of applying the electrical or electromagnetic stimulus, wave and/or one or more signals is sufficient so as to impart a metabolic change to the cells in the pre-conditioned cell inoculum, such that the metabolic change in the cells in the pre-conditioned cell inoculum is hereditary and is retained in the cells inoculated into the bioreactor or used in a subsequent biomanufacturing process or cell culture process without having to apply or reapply the electrical or electromagnetic stimulus, wave and/or one or more signals to the bioreactor or to the subsequent process.
Aspect 3: The method according to Aspect 1 or 2, wherein the cell line sample, first set of passaged cells and/or one or more subsequent sets of passaged cells includes any or any combination of cells suspended in a fluid; adherent cells; or a plurality of cells of the cell line and a liquid, cell media, substrate and/or carrier agent.
Aspect 4: The method according to Aspect 1, 2 or 3 wherein the point in the method at which the electrical or electromagnetic stimulus, wave and/or one or more signals are applied is any or any combination of: at least partly dependent on the volume or amount of the liquid, substrate, cell media and/or carrier agent in or one which the cell line sample and/or passaged cells are provided; before the first cell passage; after the first cell passage; at a last shake flask step in the method; at a last cell passage undertaken within an incubator used in the method; or at or after the third or fourth cell passage in the method.
Aspect 5: The method according to any one of Aspects 1 to 4, wherein the electrical or electromagnetic stimulus, wave and/or one or more signals are applied to the cell line sample more than or equal to between 0-12 hours, between 0-24 hours, between 0-48 hours, between 0-72 hours or between 0-96 hours before the at least first cell passage and/or before the one or more subsequent cell passages.
Aspect 6: The method according to any one of Aspects 1 to 5, wherein the cell line sample used in the method is any or any combination of a mammalian cell line, plant cell line, fungi cell line, insect cell line, bacterial cell line, fibroblast cell line, cancer cell line, 3T3 cells; A549 cells, HeLa cells, HEK 293 cells, Huh7 cells, Jurkat cells, OK cells, Ptk2 cells, Vero cells, yeast, CHO cells, PER C6 cells; Primary T cells; or Pichia cells.
Aspect 7: The method according to any one of Aspects 1 to 6, wherein the electrical or electromagnetic stimulus, wave and/or one or more signals are pulsed electromagnetic wave (PEMW) signals are at a frequency in the range of approximately 2.2-2.6 GHz+/−50 MHz, 2.4 GHz+/−50 MHz, 2.45 GHz+/−50 MHz, or within the range of the industrial, scientific and medical radio frequency band (ISM band).
Aspect 8: The method according to Aspect 7, wherein the PEMW signals are pulsed at a frequency of approximately 50 Hz or less, 25 Hz or less or 15 Hz or less.
Aspect 9: The method according to any one of Aspects 7 to 8, wherein the pulsed PEMW signals last for between Ims-20 ms; have a time period between pulses of approximately 66 ms or less; have a duty cycle of less than 2%; and/or have a power of +2 dBM-+4 dBM, approximately 1 mW, approximately 2 mW, approximately 2.5119 mW, 1 mW-2.5119 mW, 2-20 mW, or 3 dBm-13 dBm.
Aspect 10: A method for a biomanufacturing process, said method including the steps of: taking a cell line sample and undertaking at least one cell passage with said cell line sample to increase the number of cells to form at least a first set of passaged cells; and subsequently using said first set and/or one or more subsequent sets of passaged cells for inoculating a bioreactor and/or for providing a cell bank, and wherein said method further includes the step of applying an electrical or electromagnetic stimulus, wave and/or one or more signals to any or any combination of: the cell line sample, to the at least first set of passaged cells and/or one or more subsequent sets of passaged cells to create a pre-conditioned cell inoculum, subsequently inoculating the bioreactor and/or providing the cell bank with the pre-conditioned cell inoculum.
Aspect 11: A system for producing a cell inoculum suitable for use in a biomanufacturing or cell culture process, said system including at least one container for the containment of a cell line and/or at least one set of passaged cells; means for emitting an electrical or electromagnetic stimulus, wave and/or one or more signals therefrom; and wherein said means for emitting an electrical or electromagnetic stimulus, wave and/or one or more signals are arranged relative to said at least one container so that the cell line and/or the at least one set of passaged cells can receive the electrical or electromagnetic stimulus, wave and/or one or more signals in use.
Aspect 12: A cell inoculum composition suitable for use in a biomanufacturing or cell culture process comprising a population of epigenetically modified cells, wherein the cell inoculum is obtainable by a process comprising the step of applying an electrical or electromagnetic stimulus, wave and/or one or more signals to any or any combination of: a cell line sample, a first set of passaged cells derived from the cell line sample by a cell passage step to increase the number of cells, and/or one or more subsequent sets of passaged cells derived from the first set of passaged cells by one or more cell passage step(s) to increase the number of cells; wherein the epigenetically modified cells exhibit an increase in protein productivity in comparison to untreated cells, an increase in ribosomal activity in comparison to untreated cells, or both.
Aspect 13: A cell inoculum composition according to Aspect 12 wherein the epigenetically modified cells exhibit an increase in protein productivity of at least 30% or more in comparison to untreated cells, an increase in ribosomal activity of at least 50% or more in comparison to untreated cells, or both.
Aspect 14: A cell inoculum composition according to Aspect 12 or 13, wherein the electrical or electromagnetic stimulus, wave and/or one or more signals are applied to the cell line sample more than or equal to 0-12 hours, 0-24 hours, 0-48 hours, 0-72 hours or 0-96 hours before the first cell passage and/or before the one or more subsequent cell passages.
Aspect 15: A cell inoculum composition according to any one of Aspects 12 to 14, wherein the cell line sample used is any or any combination of a mammalian cell line, plant cell line, fungi cell line, insect cell line, bacterial cell line, fibroblast cell line, cancer cell line, 3T3 cells; A549 cells, HeLa cells, HEK cells including HEK 293 cells, Huh7 cells, Jurkat cells, OK cells, Ptk2 cells, Vero cells, yeast, CHO cells, PER C6 cells; Primary T cells; or fungal cells, including Pichia cells.
Aspect 16: A cell inoculum composition according to any one of Aspects 12 to 15, wherein the electrical or electromagnetic stimulus, wave and/or one or more signals are pulsed electromagnetic wave (PEMW) signals.
Aspect 17: A cell inoculum composition according to any one of Aspects 12 to 16, wherein the pulsed electromagnetic wave (PEMW) signals are at a frequency in the range of approximately 2.2-2.6 GHz+/−50 MHz, 2.4 GHz+/−50 MHz, 2.45 GHz+/−50 MHz, or within the range of the industrial, scientific and medical radio frequency band (ISM band).
Aspect 18: A cell inoculum composition according to any one of Aspects 12 to 17, wherein the PEMW signals are pulsed at a frequency of approximately 50 Hz or less, 25 Hz or less or 15 Hz or less.
Aspect 19: A cell inoculum composition according to any one of Aspects 12 to 18, wherein the epigenetically modified cells comprise HEK cells, HEK 293 cells, or CHO cells.
Aspect 20: A cell inoculum composition according to any one of Aspects 12 to 19, wherein the epigenetic modification in the said epigenetically modified cells compared to untreated cells comprises one or more of: increased ribosomal biogenesis, increased cytoplasmic ATP production or increased histone acetylation.
Aspect 21: A cell inoculum composition according to any one of Aspects 12 to 20, wherein pulsed electromagnetic wave (PEMW) signals are applied to a cell line sample following resuscitation of the cells from a cell bank.
Aspect 22: A cell inoculum composition according to any one of Aspects 12 to 21, wherein pulsed electromagnetic wave (PEMW) signals are applied to the cell inoculum, or a container comprising the cell inoculum, wherein the cell inoculum has a volume in a single vessel of 10 litres or less.
Aspect 23: A cell inoculum composition according to any one of Aspects 12 to 22 wherein the cell inoculum has a volume in a single vessel of 2 litres or less, or a volume of 1 litre or less.
Aspect 24: A cell inoculum composition obtained or obtainable by a method according to any one of Aspects 1 to 10.
Aspect 25: A cell inoculum composition comprising a population of epigenetically modified HEK cells, wherein the epigenetically modified HEK cells exhibit an increase in protein productivity in comparison to untreated cells, an increase in ribosomal activity in comparison to untreated cells, or both.
Aspect 26: A cell inoculum composition according to Aspect 25 wherein the HEK cells are HEK-293 cells.
Aspect 27: A cell inoculum composition according to Aspect 25 or 26 wherein the epigenetically modified cells exhibit an increase in protein productivity of at least 30% or more in comparison to untreated cells, an increase in ribosomal activity of at least 50% or more in comparison to untreated cells, or both.
Aspect 28: A cell inoculum composition comprising a population of epigenetically modified CHO cells, wherein the epigenetically modified CHO cells exhibit an increase in protein productivity in comparison to untreated cells, an increase in ribosomal activity in comparison to untreated cells, or both.
Aspect 29: A cell inoculum composition according to Aspect 28 wherein the epigenetically modified cells exhibit an increase in protein productivity of at least 30% or more in comparison to untreated cells, an increase in ribosomal activity of at least 50% or more in comparison to untreated cells, or both.

1. A method for producing a cell inoculum suitable for use in inoculating a bioreactor in a biomanufacturing or cell culture process; said method including the steps of taking a cell line sample and undertaking at least one cell passage with said cell line sample to increase the number of cells to form at least a first set of passaged cells; and subsequently using said first set and/or one or more subsequent sets of passaged cells for optionally inoculating a bioreactor in use; and wherein said method further includes the step of applying an electrical or electromagnetic stimulus, wave and/or one or more signals to any or any combination of: the cell line sample, the at least first set of passaged cells and/or one or more subsequent sets of passaged cells to create a pre-conditioned cell inoculum, and optionally then using the pre-conditioned cell inoculum to directly or indirectly inoculate the bioreactor.

2. The method according to claim 1, wherein the step of applying the electrical or electromagnetic stimulus, wave and/or one or more signals is sufficient so as to impart a metabolic change to the cells in the pre-conditioned cell inoculum, such that the metabolic change in the cells in the pre-conditioned cell inoculum is hereditary and is retained in the cells inoculated into the bioreactor or used in a subsequent biomanufacturing process or cell culture process without having to apply or reapply the electrical or electromagnetic stimulus, wave and/or one or more signals to the bioreactor or to the subsequent process.

3. The method according to claim 1, wherein the cell line sample, first set of passaged cells and/or one or more subsequent sets of passaged cells includes any or any combination of cells suspended in a fluid; adherent cells; or a plurality of cells of the cell line and a liquid, cell media, substrate and/or carrier agent.

4. The method according to claim 1, wherein the point in the method at which the electrical or electromagnetic stimulus, wave and/or one or more signals are applied is any or any combination of: at least partly dependent on the volume or amount of the liquid, substrate, cell media and/or carrier agent in or one which the cell line sample and/or passaged cells are provided; before the first cell passage; after the first cell passage; at a last shake flask step in the method; at a last cell passage undertaken within an incubator used in the method; or at or after the third or fourth cell passage in the method.

5. The method according to claim 1, wherein the electrical or electromagnetic stimulus, wave and/or one or more signals are applied to the cell line sample more than or equal to between 0-12 hours, between 0-24 hours, between 0-48 hours, between 0-72 hours or between 0-96 hours before the at least first cell passage and/or before the one or more subsequent cell passages.

6. The method according to claim 1, wherein the cell line sample used in the method is any or any combination of a mammalian cell line, plant cell line, fungi cell line, insect cell line, bacterial cell line, fibroblast cell line, cancer cell line, 3T3 cells; A549 cells, HeLa cells, HEK 293 cells, Huh7 cells, Jurkat cells, OK cells, Ptk2 cells, Vero cells, yeast, CHO cells, PER C6 cells; Primary T cells; or Pichia cells.

7. The method according to claim 1, wherein the electrical or electromagnetic stimulus, wave and/or one or more signals are pulsed electromagnetic wave (PEMW) signals are at a frequency in the range of approximately 2.2-2.6 GHz+/−50 MHz, 2.4 GHz+/−50 MHz, 2.45 GHz+/−50 MHz, or within the range of the industrial, scientific and medical radio frequency band (ISM band).

8. The method according to claim 7, wherein the PEMW signals are pulsed at a frequency of approximately 50 Hz or less, 25 Hz or less or 15 Hz or less.

9. The method according to claim 1, wherein the PEMW signals last for between 1 ms-20 ms; have a time period between pulses of approximately 66 ms or less; have a duty cycle of less than 2%; and/or have a power of +2 dBM-+4 dBM, approximately 1 mW, approximately 2 mW, approximately 2.5119 mW, 1 mW-2.5119 mW, 2-20 mW, or 3 dBm-13 dBm.

10. A method for a biomanufacturing process, said method including the steps of: taking a cell line sample and undertaking at least one cell passage with said cell line sample to increase the number of cells to form at least a first set of passaged cells; and subsequently using said first set and/or one or more subsequent sets of passaged cells for inoculating a bioreactor and/or for providing a cell bank, and wherein said method further includes the step of applying an electrical or electromagnetic stimulus, wave and/or one or more signals to any or any combination of: the cell line sample, to the at least first set of passaged cells and/or one or more subsequent sets of passaged cells to create a pre-conditioned cell inoculum, subsequently inoculating the bioreactor and/or providing the cell bank with the pre-conditioned cell inoculum.

11. A system for producing a cell inoculum suitable for use in a biomanufacturing or cell culture process, said system including at least one container for the containment of a cell line and/or at least one set of passaged cells; means for emitting an electrical or electromagnetic stimulus, wave and/or one or more signals therefrom; and wherein said means for emitting an electrical or electromagnetic stimulus, wave and/or one or more signals are arranged relative to said at least one container so that the cell line and/or the at least one set of passaged cells can receive the electrical or electromagnetic stimulus, wave and/or one or more signals in use.

12. A cell inoculum composition suitable for use in a biomanufacturing or cell culture process comprising a population of epigenetically modified cells, wherein the cell inoculum is obtainable by a process comprising the step of applying an electrical or electromagnetic stimulus, wave and/or one or more signals to any or any combination of: a cell line sample, a first set of passaged cells derived from the cell line sample by a cell passage step to increase the number of cells, and/or one or more subsequent sets of passaged cells derived from the first set of passaged cells by one or more cell passage step(s) to increase the number of cells; wherein the epigenetically modified cells exhibit an increase in protein productivity in comparison to untreated cells, an increase in ribosomal activity in comparison to untreated cells, or both.

13. A cell inoculum composition according to claim 12 wherein the epigenetically modified cells exhibit an increase in protein productivity of at least 30% or more in comparison to untreated cells, an increase in ribosomal activity of at least 50% or more in comparison to untreated cells, or both.

14. A cell inoculum composition according to claim 12, wherein the electrical or electromagnetic stimulus, wave and/or one or more signals are applied to the cell line sample more than or equal to 0-12 hours, 0-24 hours, 0-48 hours, 0-72 hours or 0-96 hours before the first cell passage and/or before the one or more subsequent cell passages.

15. A cell inoculum composition according to claim 12, wherein the cell line sample used is any or any combination of a mammalian cell line, plant cell line, fungi cell line, insect cell line, bacterial cell line, fibroblast cell line, cancer cell line, 3T3 cells; A549 cells, HeLa cells, HEK cells including HEK 293 cells, Huh7 cells, Jurkat cells, OK cells, Ptk2 cells, Vero cells, yeast, CHO cells, PER C6 cells; Primary T cells; or fungal cells, including Pichia cells.

16. A cell inoculum composition according to claim 12, wherein the electrical or electromagnetic stimulus, wave and/or one or more signals are pulsed electromagnetic wave (PEMW) signals.

17. A cell inoculum composition according to claim 12, wherein the pulsed electromagnetic wave (PEMW) signals are at a frequency in the range of approximately 2.2-2.6 GHz+/−50 MHz, 2.4 GHz+/−50 MHz, 2.45 GHz+/−50 MHz, or within the range of the industrial, scientific and medical radio frequency band (ISM band).

18. A cell inoculum composition according to claim 12, wherein the PEMW signals are pulsed at a frequency of approximately 50 Hz or less, 25 Hz or less or 15 Hz or less.

19. A cell inoculum composition according to claim 12, wherein the epigenetically modified cells comprise HEK cells, HEK 293 cells, or CHO cells.

20. A cell inoculum composition according to claim 12, wherein the epigenetic modification in the said epigenetically modified cells compared to untreated cells comprises one or more of: increased ribosomal biogenesis, increased cytoplasmic ATP production or increased histone acetylation.

21. A cell inoculum composition according to claim 12, wherein pulsed electromagnetic wave (PEMW) signals are applied to a cell line sample following resuscitation of the cells from a cell bank.

22. A cell inoculum composition according to claim 12, wherein pulsed electromagnetic wave (PEMW) signals are applied to the cell inoculum, or a container comprising the cell inoculum, wherein the cell inoculum has a volume in a single vessel of 10 litres or less.

23. A cell inoculum composition according to claim 22 wherein the cell inoculum has a volume in a single vessel of 2 litres or less, or a volume of 1 litre or less.

24. A cell inoculum composition obtained or obtainable by a method according to claim 1.

25. A cell inoculum composition comprising a population of epigenetically modified HEK cells, wherein the epigenetically modified HEK cells exhibit an increase in protein productivity in comparison to untreated cells, an increase in ribosomal activity in comparison to untreated cells, or both.

26. A cell inoculum composition according to claim 25 wherein the HEK cells are HEK-293 cells.

27. A cell inoculum composition according to claim 25 wherein the epigenetically modified cells exhibit an increase in protein productivity of at least 30% or more in comparison to untreated cells, an increase in ribosomal activity of at least 50% or more in comparison to untreated cells, or both.

28. A cell inoculum composition comprising a population of epigenetically modified CHO cells, wherein the epigenetically modified CHO cells exhibit an increase in protein productivity in comparison to untreated cells, an increase in ribosomal activity in comparison to untreated cells, or both.

29. A cell inoculum composition according to claim 28 wherein the epigenetically modified cells exhibit an increase in protein productivity of at least 30% or more in comparison to untreated cells, an increase in ribosomal activity of at least 50% or more in comparison to untreated cells, or both.
